(19) United States
(12) Patent Application Publication
(10) Pub. No.: US 2015/0031554 A1
(43) Pub. Date: Jan. 29, 2015

(54) EXTRACELLULAR TELOMERE ASSAY

(71) Applicant: The UAB Research Foundation, Birmingham, AL (US)

(72) Inventors: Kevin W. Harris, Birmingham, AL (US); Katri S. Selander, Birmingham, AL (US); David Graves, Birmingham, AL (US)

(21) Appl. No.: 14/373,202

(22) PCT Filed: Jan. 20, 2013
(86) PCT No.: PCT/US13/22337

§ 371 Date: Jul. 18, 2014

Related U.S. Application Data
(60) Provisional application No. 61/589,006, filed on Jan. 20, 2012.

(51) Int. Cl. C12Q 1/68

(52) U.S. Cl. CPC C12Q 1/6886; C12Q 1/6883; C12Q 2600/118
USPC 506/2; 435/6.12; 435/6.11; 506/9; 435/6.1; 435/6.14

(57) ABSTRACT

Provided herein is a method of detecting a level of cell injury in a subject, the method comprising detecting an amount of extracellular telomeres in a biological sample from the subject. The amount of extracellular telomeres as compared to a control amount indicates the level of cell injury in the subject. Further provided is a kit for detecting a level of apoptosis in a subject, the kit comprises first and second nucleic acid sequences, wherein the first and second nucleic acid sequences hybridize with an extracellular telomere; and a container. The first and second nucleic acid sequences can comprise SEQ ID NO: 1 and SEQ ID NO:2.

This application claims the benefit of U.S. Provisional Application No. 61/589,006, filed Jan. 20, 2012, which is hereby incorporated herein in its entirety.

### STATEMENT REGARDING FEDERALLY FUNDED RESEARCH

This invention was made with government funding under Department of Defense Breast Cancer Award BC095831. The government has certain rights in this invention.

### BACKGROUND

Clinical in vivo measurement of cell injury is currently indirect. There are numerous diseases associated with increased or decreased cell injury. Therefore, there is a need for efficient methods of assessing the level of cell injury in a subject.

### SUMMARY

Provided herein is a method of detecting a level of cell injury in a subject, the method comprising detecting an amount of extracellular telomeres in a biological sample from the subject. The amount of extracellular telomeres as compared to a control amount indicates the level of cell injury in the subject.

Further provided is a method of diagnosing a disease or condition associated with increased cell injury in a subject, the method comprising detecting an amount of extracellular telomeres in a biological sample from a subject with or at risk of developing a disease or condition associated with increased cell injury. The amount of extracellular telomeres correlates with the amount of cell injury.

Further provided is a method of determining the effectiveness of a treatment of a disease or condition associated with increased cell injury in a subject. The method comprises detecting an amount of extracellular telomeres in a second biological sample from a subject being treated for a disease or condition associated with increased cell injury. A decrease in the amount of extracellular telomeres in the biological sample as compared to an amount of extracellular telomeres in a first biological sample from the subject indicates the treatment is effective, and an increase or no change in the level of extracellular telomeres in the second biological sample as compared to an amount of extracellular telomeres in a first biological sample from the subject indicates the treatment is ineffective.

Also provided is a method of determining the effectiveness of a treatment (e.g., a cancer treatment) in a subject being treated to promote apoptosis, the method comprising detecting an amount of extracellular telomeres in a second biological sample from a subject being treated to promote apoptosis. An increase in the amount of extracellular telomeres in the second biological sample as compared to an amount of extracellular telomeres in a first biological sample from the subject indicates the treatment is effective, wherein a decrease or no change in the level of extracellular telomeres in the second biological sample as compared to an amount of extracellular telomeres in a first biological sample from the subject indicates the treatment is ineffective.

### DESCRIPTION OF DRAWINGS

FIG. 1 shows a graph demonstrating that extracellular telomeres are preferentially released in response to chemotherapy. Cells were treated for the indicated times with doxorubicin 10⁻⁵ M. Cell-free DNA is isolated from conditioned media. One sequence unit=amount of that DNA sequence in 1 nanogram of genomic DNA.

FIG. 2A shows a graph demonstrating extracellular telomere release by U373, a human brain cancer cell line, in response to doxorubicin treatment.

FIG. 2B shows a graph demonstrating extracellular telomere release by D54, a human brain cancer cell line, in response to doxorubicin treatment.

FIG. 3 shows a graph demonstrating that extracellular telomeres are detectable in the serum of mice in an orthotopic model of human breast cancer. MDA-MB-231 human breast cancer cells were orthotopically injected in nude mice (n=19) Animals were sacrificed at 14 days and the serum extracellular telomeres were measured. The median value is shown.

FIG. 4 shows a graph demonstrating that extracellular telomeres are detectable in the serum of patients with breast cancer. Serum was obtained from 40 breast cancer patients with a wide variety of clinical conditions in a community oncology setting. Patients have been roughly grouped into those with a history of metastatic disease and those without such a history. Other control DNA sequences are shown. Bars indicate mean values.

FIG. 5 shows a graph demonstrating DNA sequences in MDA-MB-231 cell-free conditioned media in response to doxorubicin treatment. PCR product from 1 ng of genomic DNA=1.

FIG. 6 shows an increase in telomere release in a patient undergoing chemotherapy. The bar along the X-axis indicates the days that chemotherapy is given.

FIG. 7 shows an increase in telomere release in a second patient undergoing chemotherapy. The bar along the X-axis indicates the days that chemotherapy is given.

FIG. 8 shows pyridostatin.

FIG. 9A shows that PNAs are unique in that they contain a peptide backbone with bases rather than amino acid R-groups attached.

FIG. 9B shows that PNAs can also hybridize to quadruplex DNA specifically either through Watson-Crick hybridization with a complementary strand to form a DNA/PNA duplex or through Hoogstein base pairing with a PNA quadruplex forming a hybrid PNA/DNA quadruplex

FIG. 10 shows that pyridostatin and PNAs captured two human telomeric DNA sequences (in the G-quadruplex conformation), the 22-mer (hTel-22) and the 24-mer (hTel-24).

### DETAILED DESCRIPTION

Provided herein is a method of detecting a level of cell injury in a subject. The method comprises detecting an amount of extracellular telomeres in a biological sample from the subject. The amount of extracellular telomeres as compared to a control amount indicates the level of cell injury in the subject. Optionally, treatment of the subject is initiated or changed based on the level of cell injury.

As used herein, cell injury can be, but is not limited to, programmed cell death, cell necrosis, anoikis, autophage and senescence. By way of example, the methods and kits described herein refer to apoptosis. However, this is not meant to be limiting, as these methods and kits are applicable to all forms of cell injury. Apoptosis refers to a process of programmed cell death that occurs in all multicellular organisms, in both healthy and pathological conditions, although levels can be higher or lower in certain pathological conditions. Apoptosis is characterized by biochemical events that lead to morphological changes in cells and cell death. These changes include among others, blebbing, cell shrinkage, nuclear fragmentation, chromatin condensation and chromosomal DNA fragmentation.

As used herein, a telomere(s) is a region of repetitive DNA sequence at the end of a chromosome, which protects the end of the chromosome from deterioration or from fusion with neighboring chromosomes. In response to cell death, telomeres are degraded and released from cells as extracellular telomeres. Although the release of extracellular telomeres is not limited to a specific stimulus that induces cell death, as set forth herein, telomeres are released from cancer cells in response to chemotherapy-induced cell death.

As used herein a biological sample is a sample derived from a subject and includes, but is not limited to, any cell, tissue or biological fluid. The sample can be, but is not limited to, peripheral blood, cerebrospinal fluid, ascites, plasma, urine, saliva, gastric secretion, cell culture conditioned media, a biopsy or a bone marrow specimen.

As used throughout, by subject is meant an individual. Preferably, the subject is a mammal such as a primate, and, more preferably, a human. Non-human primates include marmosets, monkeys, chimpanzees, gorillas, orangutans, and gibbons, to name a few. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Veterinary uses and formulations for same are also contemplated herein.

Any method standard in the art for quantitating nucleic acid can be utilized to detect telomeres. For example, and not to be limiting, these include quantitative PCR (qPCR), terminal restriction fragment southern blot or other gel electrophoretic techniques, a microarray, a whole sample parallel sequencing assay, structural assays such as a G-quadraplex bead assay that assesses unique telomeric G-quadraplex DNA structures (see, for example, Müller et al., “Small molecule-mediated G-quadraplex isolation from human cells,” Nat. Chem. 2(12): 1095-1098 (2010); ad Balasubramian and Neidle, “G-quadraplex nucleic acids as therapeutic targets,” Curr. Opin. Chem. Biol. 13(3): 345-53 (2009)), direct capture assays using telomere specific binding molecules such as protein-nucleic acid specific binding molecules or other sequence specific probes.

Capture assays can be performed in numerous ways. For example, single stranded biotinylated DNA that is complementary to the target sequence is mixed with the sample. A denaturing agent is added to de-hybridize duplex DNA in the sample. The denaturing agent is removed or neutralized allowing duplexes to reform while the analyte strand hybridizes to the complementary anchor strand. Streptavidin coated magnetic beads are then added to the sample allowing the anchor/target DNA hybrid to bind via a biotin/streptavidin linker. A magnetic field is then applied, immobilizing the magnetic beads while the beads are washed, removing non-target DNA and impurities. The purified target DNA can then be released from bead for further analysis.

Biotinylated strands of DNA (anchor strands) that are complementary to the target DNA strand are added to the sample mixture under denaturing conditions (this can be elevated temperature, pH, etc.). Both the anchor and target strands are unfolded under these conditions. The denaturing conditions are removed (cooled or pH adjusted) so that hybridization between the anchor and target DNA strands is achieved. To ensure complete hybridization of the target strand to the anchor as opposed to the original complementary strand, the anchor strands can be added at a large excess so that the equilibrium of target/anchor complex is favored. The streptavidin coated magnetic beads are then added to the sample, and gently mixed allowing the anchor/target hybrid to bind via a streptavidin-biotin interaction.

The magnetic beads are then immobilized to the side of the tube with a magnet and the remaining solution is removed from the sample. The hybridized sample can then be denatured again (by adjusting the temperature or pH) releasing the now purified target DNA strands from the anchor strands for further analysis.

In the methods set forth herein, the G-quadraplex capture assay can comprise contacting the sample with a biotinylated probe that is specific for a selected telomere sequence under conditions that allow binding of the probe to a selected target telomere sequence to form a complex; selecting the complex formed between the probe and the target sequence; and isolating the target telomere sequence from the complex. For example, the sample can be contacted with a biotinylated probe. Once the biotinylated probe binds the target telomere sequence, the sample can be contacted with a magnetic bead conjugated to streptavidin. Alternatively, the biotinylated probe can be bound to the magnetic bead conjugated to streptavidin prior to contacting the sample. A magnetic field can then be applied to the sample in order to select a complex formed between the probe and the target sequence. The target sequence can then be released or isolated from the complex for further analysis.

The biotinylated probe can be a nucleic acid probe, for example, a peptide nucleic acid probe that is complementary to a selected telomere sequence. The nucleic acid probe can be complementary to a 22-mer (h-Tel-22) telomeric sequence or complementary to a 24-mer (hTel-24) telomeric sequence. The nucleic acid probe can comprise SEQ ID NO: 1 or SEQ ID NO: 2. The biotinylated probe can be any biotinylated compound, for example, biotinylated pyridostatin or a derivative thereof, that specifically binds to a quadruplex structure. In the methods provided herein, the probe can be conjugated to a streptavidin coated magnetic bead.

Further, since telomeres bind multiple proteins, including the shelterin complex, assays that utilize immunologic detection of the shelterin proteins or proteins that bind telomeres can be used.

Since telomeres are repeated TTAGGG sequences of indeterminate total length, a unit called a DNA sequence unit can be utilized to determine the amount of extracellular telomeres in the methods set forth herein. One DNA sequence unit equals the amount of qPCR amplification product produced by 1 nanogram (ng) of genomic DNA template. Thus, a total amount of telomere sequence present per milliliter of biological fluid and the enrichment thereof can be quantitatively compared with the cognate genomic sequence. The amount of DNA sequence units per ng of test template DNA indicates the enrichment of the sequence in the sample. For example, a result of 50 DNA sequence units per ng of test DNA template indicates that the sequence is enriched 50-fold compared to the representation of this sequence in the genome. Thus, significant enrichment indicates the sequence of interest (e.g., telomere DNA) has been preferentially released from the cell of origin. In the methods set forth herein, the amount of extracellular telomeres can be detected by comparing the amount of extracellular telomeres with the amount of intracellular genomic telomeres per nanogram of total DNA. Detection of the amount of extracellular telomeres can be quantifying the amount of extracellular telomeres by comparing the amount of extracellular telomeres with the amount of intracellular genomic telomeres per nanogram of total DNA.

In the methods set forth herein, the amount of extracellular telomeres can be compared to a control amount. The control can be, but it not limited to, a known value, a sample from a healthy subject, a sample from a subject with an a disease associated with increased cell injury, a sample from a subject with a disease associated with decreased cell injury, a sample from a subject prior to or after undergoing treatment for a disease associated with increased cell injury, or a sample from a subject prior to or after undergoing treatment for a disease associated with decreased cell injury. The control sample can be from the same subject or from a different subject. Upon comparing the amount of extracellular telomeres in a sample from the subject with a control sample, the level of cell injury can be less than, greater than, or equal to the level of cell injury in the control sample. The level of cell injury in the sample from the subject can be less than about 10%, 20%, 30%, 40%, 50%, 60%, 70%, 80%, 90%, 100% or any percentage in between as compared to a control sample. The level of cell injury in the sample from the subject can be less than about 2-fold, 5-fold, 10-fold, 25-fold, 50-fold, 75-fold, 100-fold, 125-fold, 150-fold, 175-fold, 200-fold, 225-fold, 250-fold, 300-fold or more than the amount of extracellular telomeres in the control sample.

The level of cell injury in the sample from the subject can be greater than about 10%, 20%, 30%, 40%, 50%, 60%, 70%, 80%, 90%, 100%, 200%, 300%, 400% or more as compared to a control sample. The level of cell injury in the sample from the subject can be greater than about 2-fold, 5-fold, 10-fold, 25-fold, 50-fold, 75-fold, 100-fold, 125-fold, 150-fold, 175-fold, 200-fold, 225-fold, 250-fold, 300-fold or more than the amount of extracellular telomeres in the control sample.

The amount of extracellular telomeres can also be compared to the amount of a control sequence, for example, an actin-encoding nucleic acid sequence, a BRCA1 nucleic acid sequence, an alpha satellite nucleic acid sequence or an Alu repeat nucleic acid sequence.

With regard to detecting apoptosis, the methods of detecting a level of apoptosis provided herein can be utilized in combination with other methods for detecting apoptosis in a sample. For example, and not to be limiting, apoptosis can be measured by detecting DNA fragmentation via ELISA, gel electrophoresis or in situ cell death detection, caspase activity via fluorometric analysis, ICE protease activity via Western blotting, or membrane modification via Annexin-V staining.

In the methods set forth herein, the level of cell injury can be associated with a disease or condition or increased risk of developing a disease or condition. The disease or condition can be a disease associated with increased apoptosis or a disease associated with decreased apoptosis. Examples of diseases characterized by increased apoptosis include, but are not limited to, neurodegenerative disease (for example, Alzheimer's disease, Parkinson's disease, amyotrophic lateral sclerosis and retinitis pigmentosa), haemotologic diseases (for example, aplastic anemia, myelodysplastic syndrome, T CD4+ lymphocytopenia, G6PD deficiency), cancer, cardiovascular disease, an inflammatory disease, an infectious disease (for example, acquired immune deficiency syndrome (AIDS)), a cerebrovascular incident, trauma and a surgical disease. Examples of diseases characterized by decreased apoptosis include, but are not limited to, cancer, autoimmune diseases (for example, myastenia gravis and systemic lupus erythematosus) inflammatory diseases (bronchial asthma, inflammatory intestinal disease and pulmonary inflammation) and viral infections (for example, adenovirus and baculovirus).

The level of apoptosis can also be associated with a stage of a disease characterized by increased apoptosis or a stage of a disease characterized by decreased apoptosis. Therefore, methods of detecting a level of apoptosis in a subject can be utilized to monitor progression of a disease as well as efficacy of treatment for a disease. Patients can also be monitored for relapse or reoccurrence of a disease. Since telomere sequence can inhibit the innate immune system, methods of detecting a level of apoptosis can also be utilized to predict the risk of infection in cancer patients and other chronic disease.

As used herein, cancer is a disease in which the cells proliferate more rapidly than normal tissue growth. Cancer can be a neoplasm, also referred to as a tumor. A neoplasm can include, but is not limited to, pancreatic cancer, bladder cancer, head and neck cancer, melanoma, endometrial cancer, ovarian cancer, uterine cancer, non-Hodgkin lymphoma, breast cancer, brain cancer (e.g., glioblastoma), lung cancer, prostate cancer, colorectal cancer, thyroid cancer, renal cancer, adrenal cancer, liver cancer, neurofibromatosis, and leukemia. A neoplasm can be a solid neoplasm (e.g., sarcoma or carcinoma) or a cancerous growth affecting the hematopoietic system (e.g., lymphoma or leukemia). Other cancers include, but are not limited to neurofibromatosis.

Further provided is a method of diagnosing a disease or condition associated with increased cell injury in a subject, the method comprising detecting an amount of extracellular telomeres in a biological sample from a subject with or at risk of developing a disease or condition associated with increased cell injury, the amount of extracellular telomeres correlating with the amount of cell injury. For example, the cell injury can be, but is not limited to, apoptosis.

The amount of extracellular telomeres can be compared to the amount of extracellular telomeres in a control sample for which an amount of apoptosis is determined or known. The amount of apoptosis in a control sample can be predetermined, determined concurrently, or determined after a sample is obtained from the subject. Standards for use with the methods described herein can, for example, include data from samples from subjects without a disease associated with increased apoptosis, data from samples from subjects with a disease associated with increased apoptosis, or data from subjects at a particular stage of a disease associated with increased apoptosis. Comparisons can be made to multiple standards. The standards can be run in the same assay or can be known standards from a previous assay. For example, and not to be limiting, if the sample from the subject has an amount of extracellular telomeres that is about the same as the amount of extracellular telomeres in a control sample from a subject that does not have a disease associated with increased apoptosis, the subject is not diagnosed with a disease associated with increased apoptosis. However, if the sample from the subject has an amount of extracellular telomeres that is about the same as the amount of extracellular telomeres in a control sample from a subject with a disease associated with increased apoptosis, the subject can be diagnosed with a disease associated with increased apoptosis.

As used herein a subject at risk of developing a disease or condition associated with increased apoptosis refers to a subject who currently has a disease associated with increased apoptosis, a subject who previously has had a disease associated with increased apoptosis, or a subject at risk of developing a disease associated with increased apoptosis. A subject at risk of developing a disease associated with increased apoptosis can be genetically predisposed to a disease, e.g., a family history or have a mutation in a gene that causes the disease. Alternatively, a subject at risk for developing a disease associated with increased apoptosis can show early signs or symptoms. A subject currently with a disease associated with increased apoptosis has one or more of the symptoms of the disease and may have been diagnosed with a disease associated with increased apoptosis.

Further provided is a method of diagnosing a disease or condition associated with decreased cell injury in a subject, the method comprising detecting an amount of extracellular telomeres in a biological sample from a subject with or at risk of developing a disease or condition associated with decreased cell injury, the amount of extracellular telomeres correlating with the amount of cell injury. For example, the cell injury can be, but is not limited to, apoptosis.

The amount of extracellular telomeres can be compared to the amount of extracellular telomeres in a control sample for which an amount of apoptosis is determined or known. The amount of apoptosis in a control sample can be predetermined, determined concurrently, or determined after a sample is obtained from the subject. Standards for use with the methods described herein can, for example, include data from samples from subjects without a disease associated with decreased apoptosis, data from samples from subjects with a disease associated with decreased apoptosis, or data from subjects at a particular stage of a disease associated with decreased apoptosis. Comparisons can be made to multiple standards. The standards can be run in the same assay or can be known standards from a previous assay. For example, and not to be limiting, if the sample from the subject has an amount of extracellular telomeres that is about the same as the amount of extracellular telomeres in a control sample from a subject that does not have a disease associated with decreased apoptosis, the subject is not diagnosed with a disease associated with increased apoptosis. However, if the sample from the subject has an amount of extracellular telomeres that is about the same as the amount of extracellular telomeres in a control sample from a subject with a disease associated with decreased apoptosis, the subject can be diagnosed with a disease associated with decreased apoptosis.

As used herein a subject at risk for developing a disease or condition associated with decreased apoptosis refers to a subject who currently has a disease associated with decreased apoptosis, a subject who previously has had a disease associated with decreased apoptosis, or a subject at risk of developing a disease associated with decreased apoptosis. A subject at risk of developing a disease associated with decreased apoptosis can be genetically predisposed to a disease, e.g., a family history or have a mutation in a gene that causes the disease. Alternatively a subject at risk of developing a disease associated with decreased apoptosis can show early signs or symptoms. A subject currently with a disease associated with decreased apoptosis has one or more of the symptoms of the disease and may have been diagnosed with a disease associated with decreased apoptosis.

The methods set forth herein further comprise treating the subject diagnosed with a disease or condition associated with increased or decreased apoptosis with an effective amount of a pharmaceutical composition. As used herein the terms treatment, treat, treating or ameliorating refers to a method of reducing the effects of a disease or condition or symptom of the disease or condition. Thus in the disclosed methods, treatment can refer to a 10%, 20%, 30%, 40%, 50%, 60%, 70%, 80%, 90%, or 100% reduction or amelioration in the severity of an established disease or condition or symptom of the disease or condition. For example, the method is considered to be a treatment if there is a 10% reduction in one or more symptoms of the disease in a subject as compared to control. Thus the reduction can be a 10, 20, 30, 40, 50, 60, 70, 80, 90, 100%, or any percent reduction in between 10 and 100 as compared to native or control levels. It is understood that treatment does not necessarily refer to a cure or complete ablation of the disease, condition, or symptoms of the disease or condition.

Also provided is a method of determining the effectiveness of a cancer treatment in a subject being treated for cancer. The method comprises detecting an amount of extracellular telomeres in a second biological sample from a subject being treated for cancer. An increase in the amount of extracellular telomeres in the second biological sample as compared to an amount of extracellular telomeres in a first biological sample from the subject indicates the treatment is effective, wherein a decrease or no change in the level of extracellular telomeres in the second biological sample as compared to an amount of extracellular telomeres in a first biological sample from the subject indicates the treatment is ineffective.

Further provided is a method of determining the effectiveness of a treatment of a disease or condition associated with increased cell injury in a subject. The method comprises detecting an amount of extracellular telomeres in a second biological sample from a subject being treated for a disease or condition associated with increased cell injury. A decrease in the amount of extracellular telomeres in the biological sample as compared to an amount of extracellular telomeres in a first biological sample from the subject indicates the treatment is effective, and an increase or no change in the level of extracellular telomeres in the second biological sample as compared to an amount of extracellular telomeres in a first biological sample from the subject indicates the treatment is ineffective.

The method optionally further comprises modifying a treatment regimen of the subject based on the results of the method. The treatment regimen can be modified by increasing or decreasing the frequency and/or dosage of the treatment. The treatment regimen can also be modified by combining the current treatment with one or more additional treatments. The treatment regimen can also be modified by suspending the current treatment and starting a new treatment regimen or suspending treatment all together.

Also provided is a method of determining the effectiveness of a cancer treatment in a subject being treated for cancer. The method comprises detecting an amount of extracellular telomeres in a second biological sample from a subject being treated for cancer. An increase in the amount of extracellular telomeres in the second biological sample as compared to an amount of extracellular telomeres in a first biological sample from the subject indicates the treatment is effective, and a decrease or no change in the level of extracellular telomeres in the second biological sample as compared to an amount of extracellular telomeres in a first biological sample from the subject indicates the treatment is ineffective.

The method optionally further comprises modifying a treatment regimen of the subject based on the results of the method. The treatment regimen can be modified by increasing or decreasing the frequency and/or dosage of the cancer treatment. The treatment regimen can also be modified by combining the current treatment with one or more additional cancer treatments. The treatment regimen can also be modified by suspending the current treatment and starting a new treatment regimen or suspending treatment all together.

The cancer treatment can be, but is not limited to, one or more of chemotherapy, radiotherapy, surgery, hormone therapy, and/or immunotherapy. Chemotherapeutic agents that can be utilized to inhibit tumor growth include, but are not limited to, antineoplastic agents such as Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; 5-Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin C; Mitosper; Mitotane; Mitoxantrone; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Further examples of anti-neoplastic compounds include 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anthracyclines; anti-dorsalizing morphogenetic protein-1; antiandrogens, prostatic carcinoma; antiestrogens; antineoplastons; antisense oligonucleotides; aphidicolin glycinate; apoptosis gene modulators; apoptosis regulators; apurinic acid; ara-CDP-DL-PTBA; arginine deaminase; aromatase inhibitors; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hormone therapies; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LHRH analogs; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; progestational agents; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; Rh retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; erythrocyte gene therapy; velaresol; veramine; verdins; verteporfin; vinorelbine; vinxaltine; vitaxin; vorozole; zanoterone; zeniplatin; zilascorb; and zinostatin stimalamer.

The agents described herein can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia Pa., 2005. Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Administration can be carried out using therapeutically effective amounts of the agents described herein for periods of time effective to treat or reduce recurrence of a disease, for example, cancer. The effective amount may be determined by one of ordinary skill in the art and includes exemplary dosage amounts for a mammal of from about 0.5 to about 200 mg/kg of body weight of active compound per day, which may be administered in a single dose or in the form of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, or about 5 mg/kg of body weight of active compound per day.

According to the methods taught herein, the subject is administered an effective amount of the agent. The terms effective amount and effective dosage are used interchangeably. The term effective amount is defined as any amount necessary to produce a desired physiologic response. Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the activity of the specific compound employed, the metabolic stability and length of action of that compound, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

Any appropriate route of administration may be employed, for example, parenteral, intravenous, subcutaneous, intramuscular, intraventricular, intracorporeal, intraperitoneal, rectal, or oral administration. Administration can be systemic or local. Pharmaceutical compositions can be delivered locally to the area in need of treatment, for example by topical application or local injection. Multiple administrations and/or dosages can also be used. Effective doses can be extrapolated from dose-response curves derived from in vitro or animal model test systems.

Further provided is a kit for detecting a level of apoptosis in a subject, the kit comprises first and second nucleic acid sequences, wherein the first and second nucleic acid sequences hybridize with an extracellular telomere; and a container. The first and second nucleic acid sequences can comprise SEQ ID NO: 1 (ACACTAAGGTTTGGGTTTGGGTTTGGGTTTGGGTTAGTGT) and SEQ ID NO: 2 (TGTTAGGTATCCCTATCCCTATCCCTATCCCTATCCCTAACA), respectively. The kit can further comprise a third and fourth nucleic acid sequence capable of hybridizing with a control nucleic acid sequence. The control nucleic acid can be, but is not limited to, a control nucleic acid selected from the group consisting of an actin-encoding nucleic acid sequence, a BRCA1 nucleic acid sequence, an alpha satellite nucleic acid sequence, and an Alu repeat nucleic acid sequence. The kit can further comprise G-quadruplex binding beads.

Throughout this application, various publications are referenced. The disclosures of these publications in their entireties are hereby incorporated by reference into this application.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the compounds and/or methods claimed herein are made and evaluated, and are intended to be purely exemplary of the invention and are not intended to limit the scope of what the inventors regard as their invention except as and to the extent that they are included in the accompanying claims.

### EXAMPLES

Clinical in vivo measurement of apoptosis is currently indirect. Standard clinical measurements for response to chemotherapy involve radiologic measurements of tumor response with x-rays, CT, MRI, or PET scans. The result is more expense, time, and risk to the patient than a blood test. Radiologic imaging changes in tumors can take weeks to become apparent, whereas the results set forth herein show changes in extracellular telomere concentrations just hours after chemotherapy. In addition, a blood test for apoptosis could be done more frequently (for instance every cycle of chemotherapy, or even every day of a multiday regimen) to allow a better analysis of clinical trends.

Several biomarkers are used clinically for monitoring specific tumors (e.g. mucin-based antigens for breast cancer, prostate specific antigen for prostate cancer). However, these tests primarily measure tumor mass rather than directly assay apoptosis, and are not generally applicable to a wide range of uses

Telomeres are repeated DNA sequences that cap the ends of each chromosome in all multicellular animals. They prevent chromosome shortening during DNA replication. Telomeres are rapidly degraded during cell death. As disclosed herein, telomeres are specifically released from cancer cells in response to chemotherapy-induced cell death/apoptosis. These extracellular telomeres have not previously been measured. The methods set forth herein were utilized to measure extracellular telomeres in breast cancer patients and it was found that some patients have very high amounts. This provides a novel way to detect and measure tumors in cancer patients and allows for a simple and inexpensive blood test that could supplement or substitute the use of X-rays or CT scans for the treatment of cancer. The assay is also useful for diagnosing and treating other diseases such as cardiovascular diseases, inflammatory diseases, trauma and other surgical diseases, infectious diseases, degenerative neurologic diseases and other diseases of aging.

As set forth above, telomeres are specifically released from cancer cells in response to chemotherapy-induced apoptosis. In addition, in some cancer patients these soluble, non-cell associated telomeres, or extracellular telomeres, can be enriched in serum compared to their representation in the genome. Genomic cellular telomeres have been studied. However, extracellular telomeres have not previously been quantified or studied.

As shown herein, extracellular telomere concentrations are greatly increased and enriched following apoptotic stimuli such as chemotherapy. The disclosed studies are the first to measure the concentration of extracellular telomeres in any system. Further, in order to measure the telomere extracellular DNA concentration, it is compared to the amount of intracellular genomic telomeres on a per ng of total DNA basis. This is in contrast to cell-free DNA studies that routinely use housekeeping genes in the cell-free DNA as a basis of comparison. This method is non-informative in regard to enrichment of the DNA of interest.

### Methods

The samples to be tested were centrifuged at 1200×g for 6 minutes to remove residual cells. The supernatant was removed and centrifuged at 12,000× for 12 minutes to remove cellular debris. DNA was isolated from the final supernatant using a Qiagen DNA isolation kit. The DNA concentration of each sample was determined with the highly sensitive pico-green fluorometric assay (Invitrogen; Carlsbad, Calif.). This differential centrifugation method does not remove microvesicle particles which might contain DNA. Thus, the extracellular telomere assay also measures microvesicles that may contain telomeres. It is possible that the telomeric DNA being measured is contained in a microvesicle population called apoptotic bodies.

The obtained purified cell-free DNA was assayed for extracellular telomere sequences. Extracellular telomeres were measured using quantitative PCR (qPCR) with the widely accepted method of Cawthon (“Telomere measurement by quantitative PCR,” Nucleic Acids Research, 30:e47 (2002)). Other DNA sequences (actin, BRCA 1, alpha satellite DNA, and Alu repeats) were analyzed using published primers and conditions. Normal human genomic DNA (Roche; Basel, Switzerland) was used to create a telomere qPCR standard curve for the clinical specimens. For mouse standard curves, normal mouse genomic DNA isolated from murine peripheral blood cells was used. For cell culture experiments standard curves were constructed with the genomic DNA isolated from the cell line being studied.

Since telomeres are massively repeated TTAGGG sequences of indeterminate total length, the concept of gene amplification for this sequence has no useful meaning. Thus, a simple unit called a DNA sequence unit was developed. One DNA sequence unit equals the amount of qPCR amplification product produced by 1 ng of genomic DNA template. This unit is not directly a function of telomere length. Thus, one of skill in the art can comparatively quantify a total amount of telomere sequence (or any other DNA sequence) present per ml of any biological fluid, and the enrichment thereof compared to the cognate genomic sequence. The amount of DNA sequence units per ng of test template DNA indicates the enrichment of the sequence in the sample. For instance, a result of 50 DNA sequence units per ng of test DNA template indicates that sequence is enriched 50-fold compared to the representation of this sequence in the genome. Thus, significant enrichment indicates the sequence of interest (e.g. telomere DNA) has been preferentially released from the cell of origin.

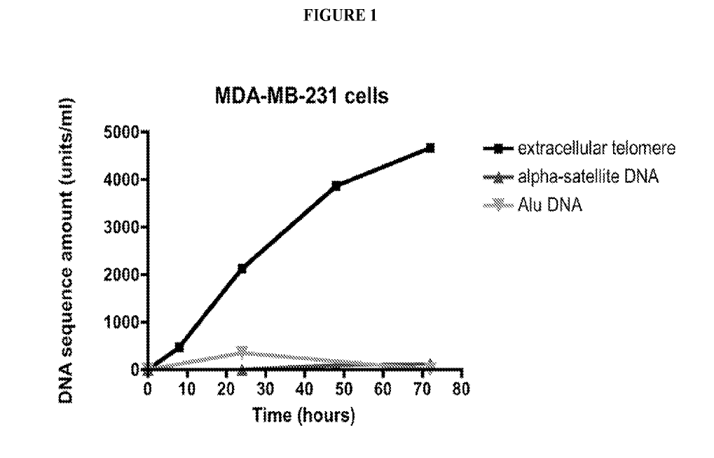

FIG. 1 shows that extracellular telomeres are specifically released from human MDA-MB-23 1 breast cancer cells following treatment with doxirubicin chemotherapy. Alu sequence was selected as a control for release of non-specific background genomic DNA. Alu sequences are randomly repeated throughout the human genome and comprise about 10% of the entire human genome. Alpha satellite sequences were selected as a representative genomic non-coding structural DNA sequence. Alpha satellite DNA is from the chromosomal centromere.

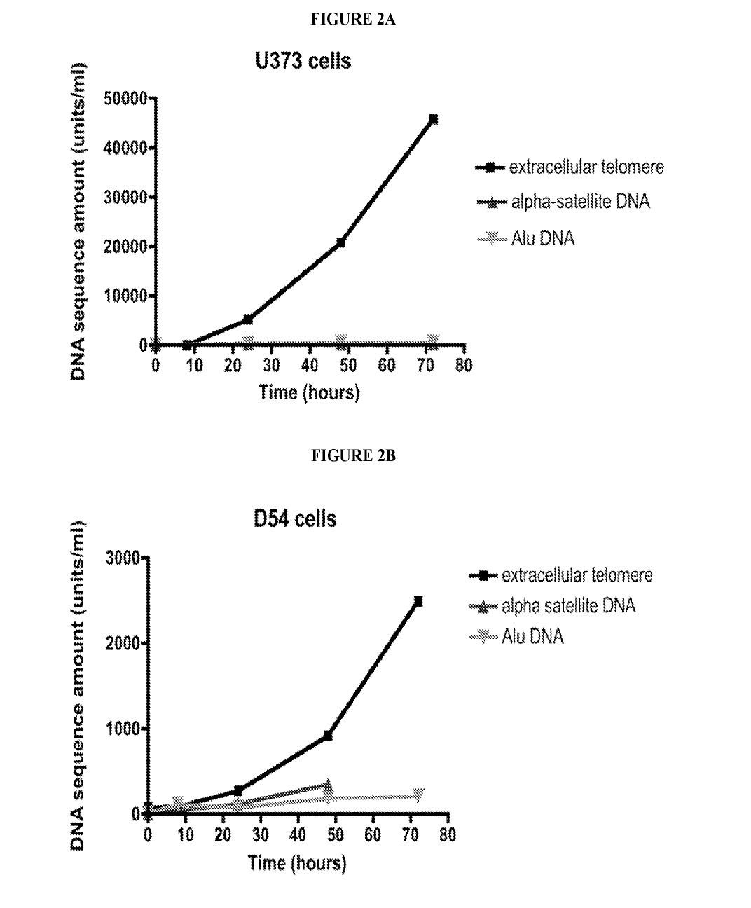

FIGS. 2A and B show similar results to FIG. 1 using human brain cancer cell lines. Similar results were also obtained with human mesothelioma cancer cell line H-meso.

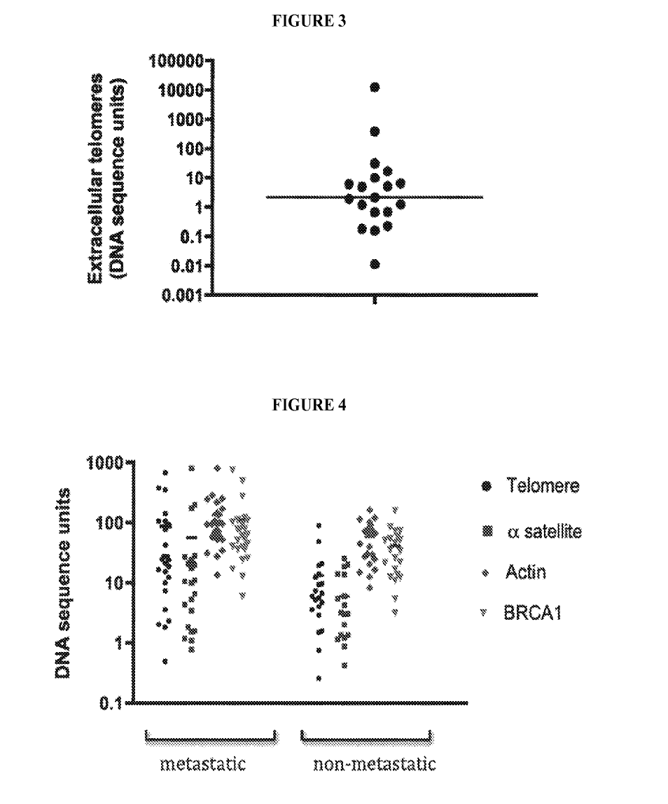

FIG. 3 demonstrates that extracellular telomeres can be detected in tumor-bearing nude mice in an orthotopic model of human breast cancer. FIG. 3 also shows a range of extracellular telomere concentrations, spanning over 4 orders of magnitude. This is likely related to varying degrees of necrosis in the individual tumors.

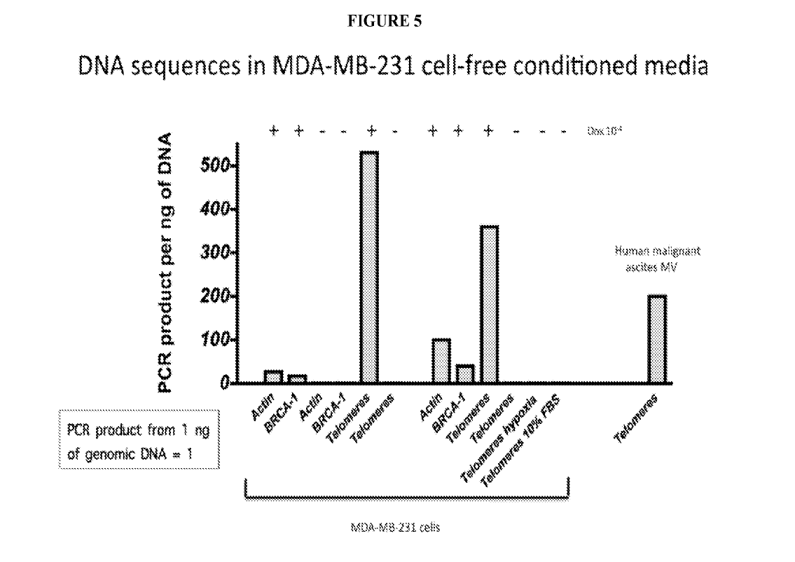

FIG. 4 shows that extracellular telomeres are also detectable in patients with breast cancer. The patients were selected randomly from a community oncology setting. The only goal of these measurements was to determine if extracellular telomeres are present in clinical samples. The groups were not assigned to test any clinical hypothesis, and are just grouped into metastatic and non-metastatic for interest. There are patients in both groups receiving chemotherapy, and also patients in both groups not receiving chemotherapy.

FIG. 5 shows a graph demonstrating DNA sequences in MDA-MB-231 cell-free conditioned media in response to doxorubicin treatment. PCR product from 1 ng of genomic DNA=1. Quantitative PCR was used to measure telomeres and genomic sequences in conditioned media from the MDA-MB-231 breast cancer cell line. Results demonstrate that telomeres were highly enriched in media from breast cancer cells treated with doxorubicin (but not untreated cells, or cells subjected to hypoxia) and in malignant ascites from a patient with pancreatic cancer. DNA coding regions of actin and BRCA-1 were much less enriched, as were c-myc and HER-s genes. The results of 2 independent experiments are shown.

### Extracellular Telomeres in Patients Undergoing Chemotherapy for Acute Myeloid Leukemia

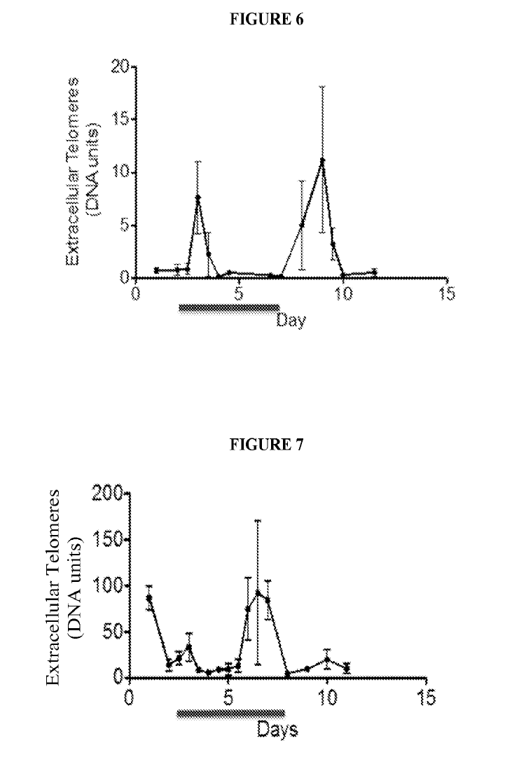

A clinical trial showing that extracellular telomeres are released into the blood of cancer patients following chemotherapy was completed. 20 patients receiving in-patient chemotherapy for acute myeloid leukemia (AML) at UAB Hospital were enrolled. Blood was obtained every 12-24 hours prior to, during, and after chemotherapy with doxorubicin and cytarabine. Serum was prepared for extracellular telomere measurements. FIGS. 6 and 7 show typical results obtained with 2 of the patients. All the patients analyzed show an obvious increase of extracellular telomeres within about 24 hours of starting chemotherapy. Most patients show 2 separate peaks of telomere release. This most likely indicates 2 populations of cells with differing sensitivity to the chemotherapy. This trial demonstrates that extracellular telomeres are released in response to chemotherapy in cancer patients.

### G-Quadruplex Capture Extracellular Telomere Bead Assay

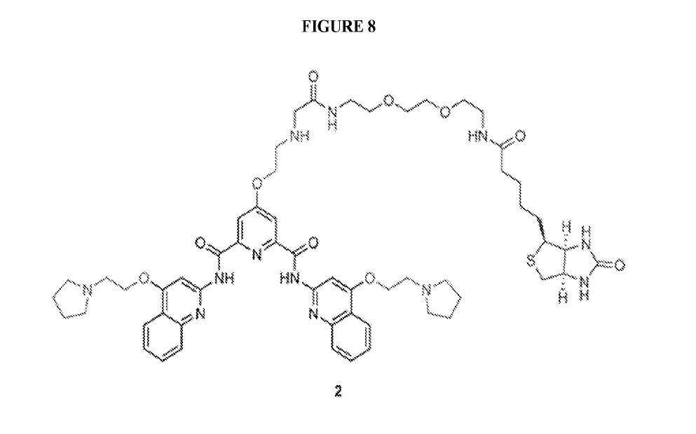

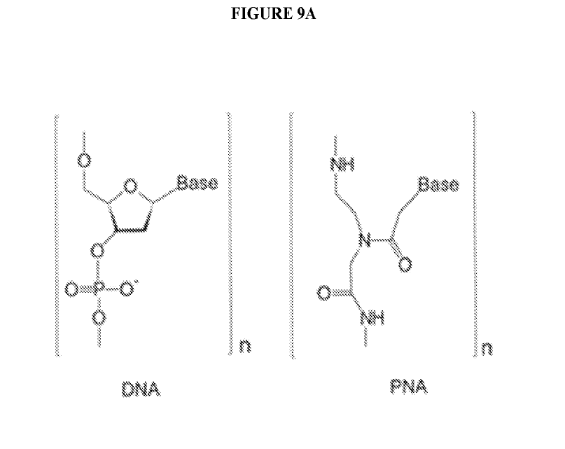

The method of G-quadruplex bead capture assay as described by Balasubramanian et al. (Curr. Opin. Chem. Biol. 13(3): 345-53 (2009)) can be adapted to measure extracellular telomeres. Pyridostatin (FIG. 8), derived from N,N′-bis(2-quinoinyl)pyridine-2,6-dicarboxamide, specifically binds to any quadraplex forming nucleic acid based on structural recognition. Pyridostatin is synthesized with a hydrophilic linker and biotin affinity-tag. Streptavidin coated magnetic beads (Streptavidin MagneSphere™ paramagnetic particles from Promega® (Madison, Wis.)) are used to link pyridostatin to the bead, providing an immobilized capture molecule specific for G-quadruplex structural motifs. This molecule, when attached to magnetic beads through a biotin/streptavidin linkage, binds exclusively quadraplex DNA or RNA regardless of the specific sequence.

This pyridostatin-linked magnetic bead is selective for G-quadruplex capture over other DNA structural motifs including duplex, single strand, and hairpins. The pyridostatin loaded beads have the capacity to selectively pull down G-quadruplex from solution and subsequent recovery of target DNA from the beads through heating and/or denaturating agents such as salts or urea is possible. Moreover, this methodology is effective in binding G-quadruplex forming sequences of over 100 nucleotides in length, indicating that it could be effective in sequestering genomic DNA fragments containing h-telomere sequences. The G-quadruplex capture bead can be used for selective pulldown of telomere sequences from DNA isolated from human serum and other bodily fluids as listed above. Captured DNA can be eluted and quantified by pico-green or other common methods, thereby eliminating the need to perform extracellular telomere qPCR. Alternatively, extracellular qPCR can be performed on the eluted DNA and the depleted DNA to ensure complete recovery of telomeres. Quantitation of the telomeric DNA can also be accomplished through fluorescence (FRET). Another application of this technique is to pull-down G-quadruplex structures directly from clinical samples without DNA isolation. In conjunction with measurement of purified DNA per above, this allows assessment of the fraction of extracellular telomeres in the G-quadruplex vs. alpha helix structure. (Nuclear regulatory proteins bound to the extracellular telomeres may inhibit G-quadruplex formation). This technique specifically detects cancer-derived extracellular telomeres mixed with healthy cell derived extracellular telomeres in patients with minimal residual disease.

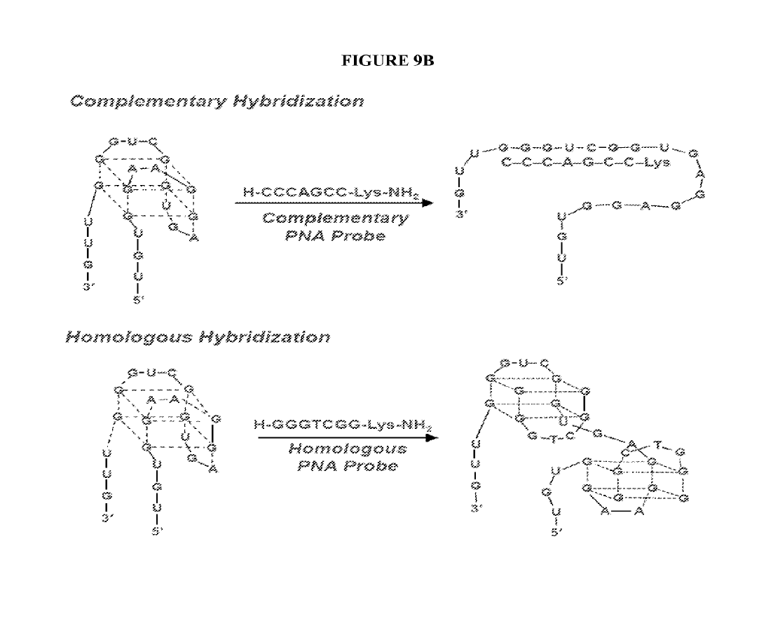

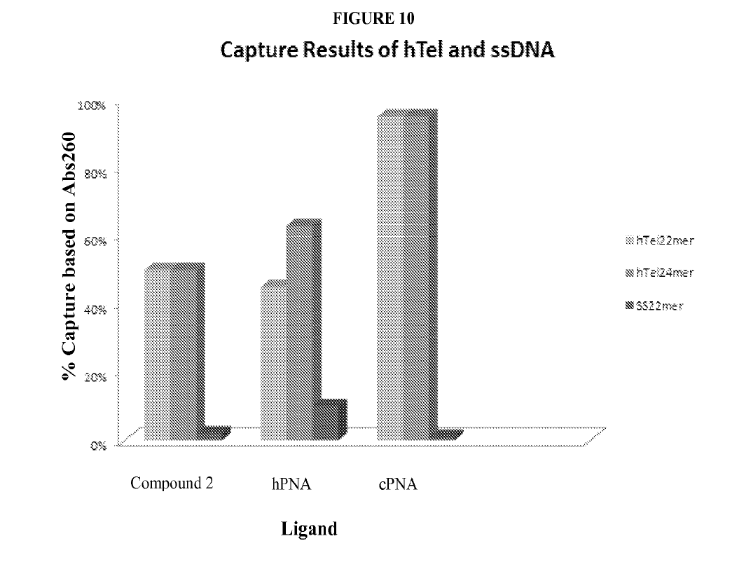

An alternative to using pyridostatin or a derivative thereof, is the use of a peptide nucleic acid as an anchor molecule. As seen in FIG. 9A, instead of the sugar-phosphate backbone as found in native nucleic acids, PNAs are unique in that they contain a peptide backbone with bases rather than amino acid R-groups attached. PNA strands can form Watson-Crick basepairs with DNA or RNA strands with remarkably high affinity and are nuclease and protease resistant. Of particular interest is that PNAs can also hybridize to quadruplex DNA specifically either through Watson-Crick hybridization with a complementary strand to form a DNA/PNA duplex or through Hoogstein base pairing with a PNA quadruplex forming a hybrid PNA/DNA quadruplex as seen in FIG. 9B. Utilization of biotinylated homologous PNA allows binding and capturing of any quadruplex forming sequence within a mixture of nucleic acids. Once the capture sequence is purified and released, the released strand can be studied further. Once the sequence of a target DNA is known, a biotinylated strand complimentary to the sequence can be used to isolate specific quadruplex forming (or any other structural motif) sequences. As shown in FIG. 10, PNAs were used to capture two human telomeric DNA sequences (in the G-quadruplex conformation), the 22-mer (hTel-22) and the 24-mer (hTel-24). The control DNA (ss-22) sequence is identical to that of the h-Tel-22 except that in the GGG repeat, the second G is mutated to an A, so that it cannot form a G-quadruplex DNA. Three different “capture” ligands were used. Two of the ligands, pyridostatin and homologues PNA (hPNA), can bind to quadruplex DNA in a structural specific manner without sequence specificity. The third “capture ligand” ligand, complimentary PNA (cPNA), is a complimentary sequence to the human telomere sequence and will hybridize to hTel DNA to form a duplex. Three different target strands were used to test the structural and sequence specificity of the ligands. Two sequences, hTel22mer and hTel24mer, are both quadruplex forming human telomere sequences (5′-(GGGTTA)ₙ-3′) that differ in the number of nucleotides on the single stranded 5′ end. The ss22mer is a single strand sequence derived from the hTel sequence where the middle guanine in the G-run is mutated to adenine as to disrupt the quadruplex fold forcing it to a single strand structure. In all assays, the biotinated ligand was first mixed with micromolar concentrations of DNA and allowed to incubate for one hour. Streptavidin coated magnetic beads were then mixed in increasing increments with the DNA/ligand mixture and allowed to shake gently for 30 minutes. After each addition of beads, the mixture was placed in the magnet, allowing the beads to stick to the side of the vial while the supernatant was analyzed by the absorbance at 260 nm in the UV-Vis spectrophotometer. This was repeated until the absorbance no longer changed upon the addition of beads. As can be seen in FIG. 10, the complimentary PNA gave the best capture efficiency for both the 22mer and 24mer human telomere sequences (>95%) while both the structurally specific ligands, compound 2 and hPNA, were able to capture the quadruplex sequences with at least 50% efficiency.

1. A method of detecting a level of cell injury in a subject, the method comprising
detecting an amount of extracellular telomeres in a biological sample from the subject, wherein the amount of extracellular telomeres as compared to a control amount indicates the level of cell injury in the subject.

2. (canceled)

3. The method of claim 1, wherein the amount of extracellular telomeres is detected by comparing the amount of extracellular telomeres with the amount of intracellular genomic telomeres per nanogram of total DNA.

4.-6. (canceled)

7. The method of claim 1, wherein detecting an amount of extracellular telomeres comprises utilizing an assay selected from the group consisting of quantitative polymerase chain reaction (qPCR), terminal restriction fragment southern blot, a gel electrophoretic assay, a structural assay, a direct capture assay, a microarray assay, and a whole sample parallel sequencing assay.

8.-10. (canceled)

11. The method of claim 1, wherein the level of cell injury is associated with a disease or condition selected from the group consisting of cancer, cardiovascular disease, an inflammatory disease, an infectious disease, a neurodegenerative disease or condition, trauma and a surgical disease.

12.-13. (canceled)

14. A method of diagnosing a disease or condition associated with increased cell injury in a subject, the method comprising: detecting an amount of extracellular telomeres in a biological sample from a subject with or at risk of developing a disease or condition associated with increased cell injury, the amount of extracellular telomeres correlating with the amount of cell injury.

15. (canceled)

16. The method of claim 14, wherein the amount of extracellular telomeres is detected by comparing the amount of extracellular telomeres with the amount of intracellular genomic telomeres per nanogram of total DNA.

17. The method of claim 14, wherein the disease or condition associated with increased cell injury is selected from the group consisting of a cancer, cardiovascular disease, an inflammatory disease, an infectious disease, a neurodegenerative disease or condition, trauma, and a surgical disease.

18.-20. (canceled)

21. The method of claim 14, wherein detecting a level of extracellular telomeres comprises utilizing an assay selected from the group consisting of quantitative polymerase chain reaction (qPCR), terminal restriction fragment southern blot, a gel electrophoretic assay, a structural assay, a direct capture assay, a microarray assay, and a whole sample parallel sequencing assay.

22.-24. (canceled)

25. A method of determining the effectiveness of a cancer treatment in a subject being treated for cancer, the method comprising detecting an amount of extracellular telomeres in a second biological sample from a subject being treated for cancer, wherein an increase in the amount of extracellular telomeres in the second biological sample as compared to an amount of extracellular telomeres in a first biological sample from the subject indicates the treatment is effective, and wherein a decrease or no change in the level of extracellular telomeres in the second biological sample as compared to an amount of extracellular telomeres in a first biological sample from the subject indicates the treatment is ineffective.

26. The method of claim 25, wherein the amount of extracellular telomeres is detected by comparing the amount of extracellular telomeres with the amount of intracellular genomic telomeres per nanogram of total DNA.

27.-29. (canceled)

30. The method of claim 1, wherein detecting a level of extracellular telomeres comprises utilizing an assay selected from the group consisting of quantitative polymerase chain reaction (qPCR), terminal restriction fragment southern blot, a gel electrophoretic assay, a structural assay, a direct capture assay, a microarray assay, and a whole sample parallel sequencing assay.

31.-53. (canceled)


SEQUENCE LISTING (2 sequences)

SEQ ID NO: 1 - DNA, 40 bp, Artificial Sequence
  Synthetic construct
acactaaggtttgggtttgggtttgggtttgggttagtgt

SEQ ID NO: 2 - DNA, 42 bp, Artificial Sequence
  Synthetic construct
tgttaggtatccctatccctatccctatccctatccctaaca